(19) United States
(12) Patent Application Publication
(10) Pub. No.: US 2015/0206303 A1
(43) Pub. Date: Jul. 23, 2015

(54) Method for Processing Images of Pulmonary Circulation and Device for Performing the Method

(71) Applicant: Ludwig Boltzmann Gesellschaft GMBH, Vienna (AT)

(72) Inventors: Michael Pienn, Graz (AT); Zoltan Balint, Graz (AT); Horst Olschewski, Graz (AT); Rudolf Stollberger, Graz (AT); Gabor Kovacs, Graz (AT)

(21) Appl. No.: 14/411,535

(22) PCT Filed: Jun. 25, 2013
(86) PCT No.: PCT/AT2013/050127

§ 371 Date: Dec. 29, 2014

(30) Foreign Application Priority Data
Jun. 29, 2012 (AT) 50258/2012

(51) Int. Cl. G06T 7/00; G06T 7/20

(52) U.S. Cl. CPC G06T 7/0016; G06T 7/20; G06T 2207/10088; G06T 2207/10116; G06T 2207/30061; G06T 2207/30104; A61B 5/0263

(57) ABSTRACT

The invention relates to a device (10) and method for processing images (1) of the pulmonary circulation (2) of a living being in order to characterize the arterial blood flow, wherein a plurality of temporally successive images (1) are processed, said device having a selection interface (12) containing at least two defined locations (a, b, c) of an image (1), a unit (13) for assigning the at least two defined locations (a, b, c) to all images (1), a unit (14) for determining the density (D) at at least two defined locations (a, b, c) in all images (1), a unit (14) for calculating the density (D) for the defined locations (a, b, c) as a function (Fₐ, fa, fb, fc) of time (t), a unit (16) for analysing the at least one time difference (Δb, Δc) between the maximum values (Ma, Mb, Mc) of the density (D) for the at least two defined locations (a, b, c) as a function of time (t), and a display (17) for the at least one time difference (Δb, Δc).

The invention relates to a method for processing images of the pulmonary circulation of a living being in order to characterize the arterial blood flow, wherein a plurality of temporally successive images are processed, and to a device for performing the method according to the invention.

The characterization of the arterial blood flow, in particular in the pulmonary circulation, is of great significance for the detection of pulmonary hypertension (PH), i.e. high blood pressure in the pulmonary circulation. PH is often the consequence of a chronic obstructive pulmonary disease (COPD) or of other diseases such as, for instance, cardiac insufficiency, pulmonary embolism, pulmonary fibrosis, congenital heart defect, etc., but it may also occur without recognizable causes (idiopathic pulmonary arterial hypertension, IPAH). PH is often difficult to diagnose since many of the symptoms occurring, such as fatigue, breathlessness and dizziness, also occur with other disorders. The estimated number of unreported cases of patients with pulmonary hypertension is deemed to be high. Every year, about 2 to 3 persons/1,000,000 inhabitants are diagnosed with IPAH, but this makes up for just approx. 5 percent of all the cases with PH. The mortality rate of IPAH—three years from detection—is approx. 50 percent without treatment. If the disease is detected only at a late stage, the annual treatment costs may amount to 300,000 Euro. Early detection is therefore essential. Also PH in the case of COPD, pulmonary fibrosis or cardiac insufficiency aggravates the prognosis substantially.

So far, PH has above all been detected by means of an invasive examination with the right heart catheter (Swan-Ganz catheter). In this process, a thin catheter is introduced into the cardiac vein or into another great vein of the body and guided through the right atrium of the heart and the right ventricle into the pulmonary artery (PA). In doing so, the pressure is measured continuously. With a healthy person the medium pressure in the pulmonary artery (mPAP) is 14.0±3.3 mmHg. PH is diagnosed if the mPAP reaches or exceeds 25 mmHg. The direct measurement of the pressure conditions in the PA from the outside is not possible.

A non-invasive assessment of the mPAP can be performed by means of Doppler sonography. In this process, the rate of a recurrent blood flow from the right ventricle into the right atrium is measured, and the pressure conditions are derived therefrom. However, since this recurrent flow occurs in relatively late stages of the disease only, and since in many cases unreliable estimations of the pressure are available, this method is not suited for early detection. Due to the comparatively coarse assessment this method is only used for a first screening which is followed by further examinations.

In the case of radiologic examinations of the thorax by means of radiography, computer tomography (CT) or magnetic resonance tomography (MRT) it is possible to find further indications of PH from morphology. These comprise the determination of the diameters of PA and aorta, the thickening of the cardiac muscles, in particular of the right heart, the enlargement of the right heart as such and the change of curvature of the cardiac septum due to the changed pressure conditions. By appropriate methods it is also possible to determine functional parameters with these examinations. The reduction of the flexibility of the PA with respect to the pressure changes during a heart beat (distensibility) may be used as a diagnostic parameter. However, in this case images triggered by electrocardiogram are required, which, in the case of CT examinations, result in an increased radiographic dose and/or in the case of MRT examinations in a longer duration of examination.

As is illustrated in US 2010/0094122 A1, the distribution of the blood flow rates may be measured in MRT examinations with phase contrast imaging. If increased pressure exists in the PA, a whirl will be generated in the main trunk of the PA which may be illustrated this way. It is likewise possible to measure the time during which a recurrent blood flow through the whirl exists and it may be related to the duration of the heart beat or the average flow rate in a heart beat may be measured. By means of these latter-mentioned methods it is possible to diagnose PH with MRT. Drawbacks are the effort involved with a MRT examination and the costs incurred, which exclude this kind of examination, for instance, as a routine examination or a preventive examination.

As compared to this, it is an object of the present invention to enable the examination and partial characterization of the arterial blood flow in the pulmonary circulation which is, for instance, relevant for the detection of PH, by means of non-invasive imaging methods without an invasive intervention and without time-consuming and expensive MRT examinations. Disadvantages of known methods and devices are intended to be avoided or to be at least reduced.

The method according to the invention of the kind as defined in the outset solves this object in that

a) at least two locations in an image are defined,

b) the defined locations are assigned to all images,

c) the density at the defined locations is determined in all images,

d) the density at the defined locations is calculated as a function of time, and

e) at least one time difference between the maximum values of the density at the at least two defined locations as a function of time is analysed and displayed.

The device according to the invention for solving the object indicated comprises accordingly

a) a selection interface of at least two defined locations of an image,

b) a unit for assigning the at least two defined locations to all images,

c) a unit for determining the density at at least two defined locations in all images,

d) a unit for calculating the density for the defined locations as a function of time,

e) a unit for analysing the at least one time difference between the maximum values of the density for the at least two defined locations as a function of time, and

f) a display for the at least one time difference.

The method according to the invention and/or the device according to the invention enable in an advantageous manner the non-invasive characterization of the arterial blood flow in the pulmonary circulation by using exclusively spatially resolving images. Additional information relating to the blood flow such as, for instance, the direction of movement and the rate of movement of the blood or blood pressure values are not required. Therefore, it is possible to apply the method to images of different origin, wherein the required image quality (resolution, contrast, etc.) merely depends on the size of the locations to be defined. It is not of decisive importance in which form the images are available nor by means of which method they were produced, which is why the general term of density may refer to any parameter. The density may, for instance, be the density of a coloring, a color value, a contrast value, or a radiation, i.e. the intensity thereof. Likewise, the concentration of an imaging or imaged agent, for instance, a contrast agent concentration or a hydrogen content, may be meant therewith. Furthermore, a signal density, a signal intensity, a grey value or a brightening may be comprised by the general term of density. It is to be understood that the density determined with the present method may also relate to the respectively inverse parameters of the described magnitudes (darkening, radiation absorption, etc.).

Since the required images may generally be produced without invasive intervention, in particular in the context of a routine examination, and since the method can even be performed in the absence of the living being examined (in the following briefly referred to as “patient”), the risk of infections, injuries, etc. associated with such interventions is avoided and safety is increased. Frequently, the images utilizable for the method are produced in any case in the context of an extensive examination and the taking of additional images may either be omitted completely or be reduced to a minimum, wherein no preparations of the patient exceeding those of a routine examination are required. The patient's comfort is therefore not or hardly reduced.

Particularly meaningful results can be achieved if the images of the pulmonary circulation substantially represent cuts in the region of the pulmonary trunk (Truncus pulmonalis) and at least one pulmonary artery (Arteria pulmonalis), wherein one defined locations is selected or was already arranged in the region of the pulmonary trunk and at least one defined location in the region of the at least one pulmonary artery. In the case of images of human beings, the cuts may in particular be substantially horizontal and/or transversal cuts, i.e. cuts that are approximately perpendicular to the longitudinal axis of the body and/or the spine. Thus, it is possible to examine the blood flow at the beginning of the pulmonary circulation, i.e. directly after the heart. The results determined for this region are particularly useful since a strongly differing behavior of the blood occurs here in the case of a disease and since possible deviations from the result expected in the case of a healthy patient are particularly clear to detect here. The locations defined for determining the density may be selected in the pulmonary trunk and/or in the main trunk of the pulmonary artery, on the one hand, and in a right and/or left pulmonary artery, on the other hand. Due to the anatomy of these sections of the pulmonary circulation, in particular the length of the pulmonary trunk and the pulmonary arteries, the distance achieved by such selection between the defined locations is restricted within a particular region and the time difference determined enables coarse conclusions on the blood flow rate between the defined points even without knowing the case-related exact anatomy. The selection of more than two locations, in particular of locations both in the right and in the left pulmonary arteries, offers the possibility of self-control and of a plausibility check of the method and possibly a differentiated examination of the right and left lungs without additional effort for the patient. Locations arranged further downstream in the pulmonary circulation may on principle also be selected if the image quality of the images used is sufficient to image the correspondingly smaller vessels, to select the desired locations, and to determine the density there with sufficient exactness.

Since this kind of images is either already available in most cases or is quickly and cost-efficiently to produce, radiographic images using a contrast agent can advantageously be used as images. The devices required for such images are particularly wide spread, which is why the effort for the patient is very small since high local and temporal flexibility is guaranteed due to the distribution. The use of a contrast agent which is preferably applied over a short period, for instance about 4 seconds, achieves a good temporal resolution of the method according to the invention.

If the density of the images at the defined locations is determined as average radiographic attenuation, the evaluation of the images and/or the determination of the density is particularly simple. Averaging reduces the sensitivity of the method with respect to temporary fluctuations and imaging errors and thus improves the result and/or enables the use of images with interferences. For this reason, with the device according to the invention the unit for determining the density of the images may be designed for determining the average radiographic attenuation at the defined locations.

In another advantageous variant of the invention the images may be magnetic resonance tomography images (MRT images) using a contrast agent. Such images have the advantages over the afore-described radiographic images that the contrast agents are, as a rule, better tolerable and that the method and/or the device may thus also be used for patients with intolerances of radiographic contrast agents.

When using MRT images it is beneficial to determine the average signal and/or the average brightening of the images at the defined locations with suitable weighting as the density underlying the method. In this context there apply substantially the same advantages as with the use of the average radiographic attenuation. Accordingly, the unit for determining the density of the images may be designed for determining the average signal and/or the average brightening with suitable weighting at the defined locations.

It is beneficial if images are processed at an interval between the moments of capture of at most 5 s, preferably between 0.5 and 2 s. A smaller interval indeed improves the exactness of the determined time difference in principle, but requires—due to the higher number of images—handling of larger amounts of data and is usually associated with higher exposure of the patient.

The exactness of the result, i.e. of the determined time difference, may in particular be distinctly better than the intervals of the images if the density for every defined location is interpolated as a function of time prior to the analysis of the at least one time difference between the maximum values of the density, and the time difference between the maximum values of the interpolated functions is analyzed. The unit for analyzing the at least one time difference between the maximum values of the density may comprise a module for interpolation of the density as a function of time for this purpose. An interpolation is in particular justified due to the fact that unexpected jumps in the density progress can be excluded. A spline interpolation which assesses a smooth progress of the density as a function of time has turned out to be particularly suited for these purposes.

If, in addition to the determined time difference, the distance between the defined locations is determined, it is possible to determine a blood flow rate from the distance and the time difference. The rate determined this way may, for instance, serve to be compared with the results of other examinations. Moreover, the blood flow rate is a better reproducible and thus more meaningful result since the dependency of the value determined in this way on the respective anatomy of the patient is smaller. In the device according to the invention a unit for determining the distance between the defined locations and for determining a blood flow rate from the distance and the time difference may therefore be connected advantageously with the unit for analyzing the at least one time difference.

In order to draw the attention of a user to an unexpected result, the at least one time difference may be compared with at least one predetermined limit value, preferably approx. 0.5 s, and a signal may be output if the at least one limit value is exceeded. For this purpose, the display of the device according to the invention may comprise a signaler for comparing the time difference with a predetermined limit value, preferably approx. 0.5 s, and for outputting a signal if the limit value is exceeded. It is then the user's task to determine the reason for the unusually high time difference and to possibly verify the selection of the defined locations or to repeat the method. If procedural errors can be excluded, a time difference of more than 0.5 s may, for instance, be indicative of the existence of PH, which may subsequently be verified by means of specific examinations. In the case of a likewise determined blood flow rate, the latter may in analogy be compared with an own limit value, preferably approx. 120 mm/s, and a signal may be output if the limit value is undercut.

The invention will be described further in the following by means of particularly preferred embodiments, which it is not intended to be restricted to, though, and with reference to the drawing. In the drawing there show in detail:

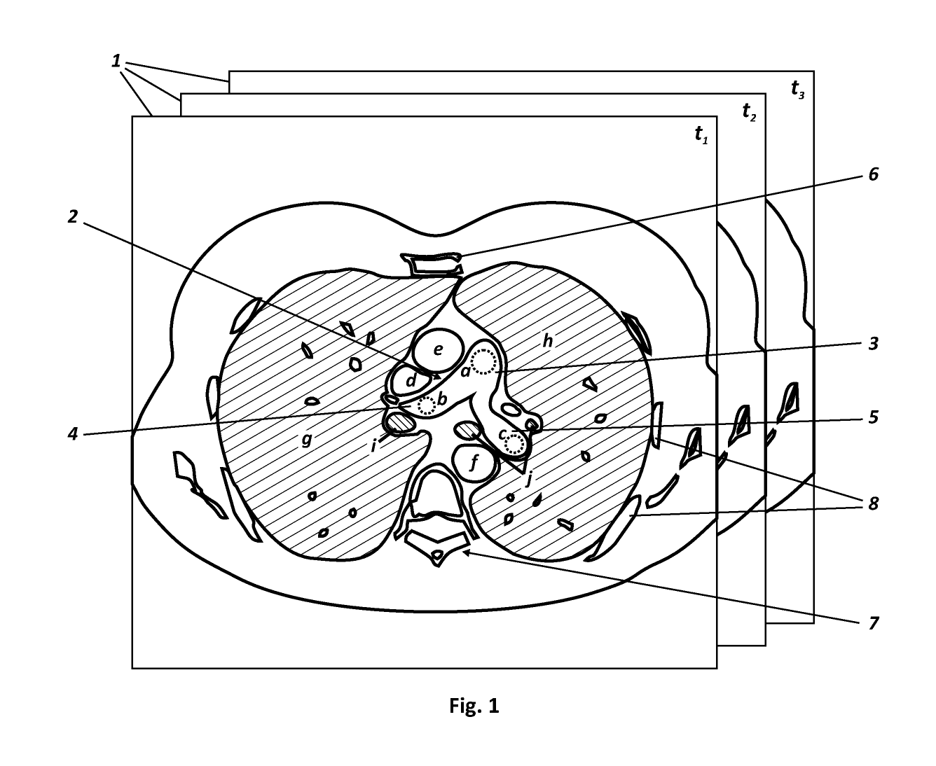

FIG. 1 schematically a series of sectional images of a human thorax;

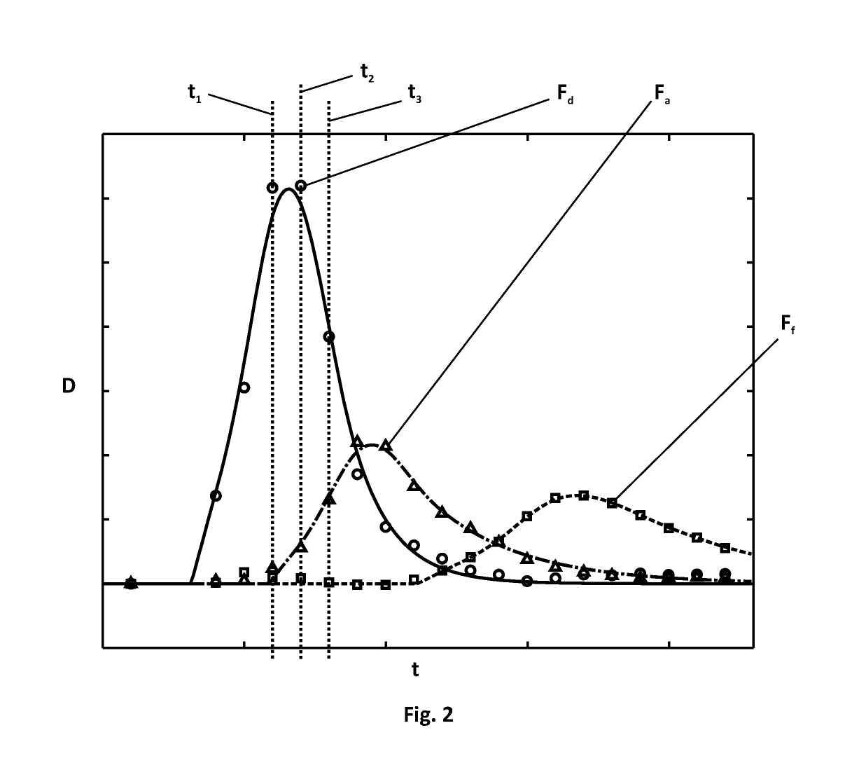

FIG. 2 the density at three locations pursuant to FIG. 1 as a function of time;

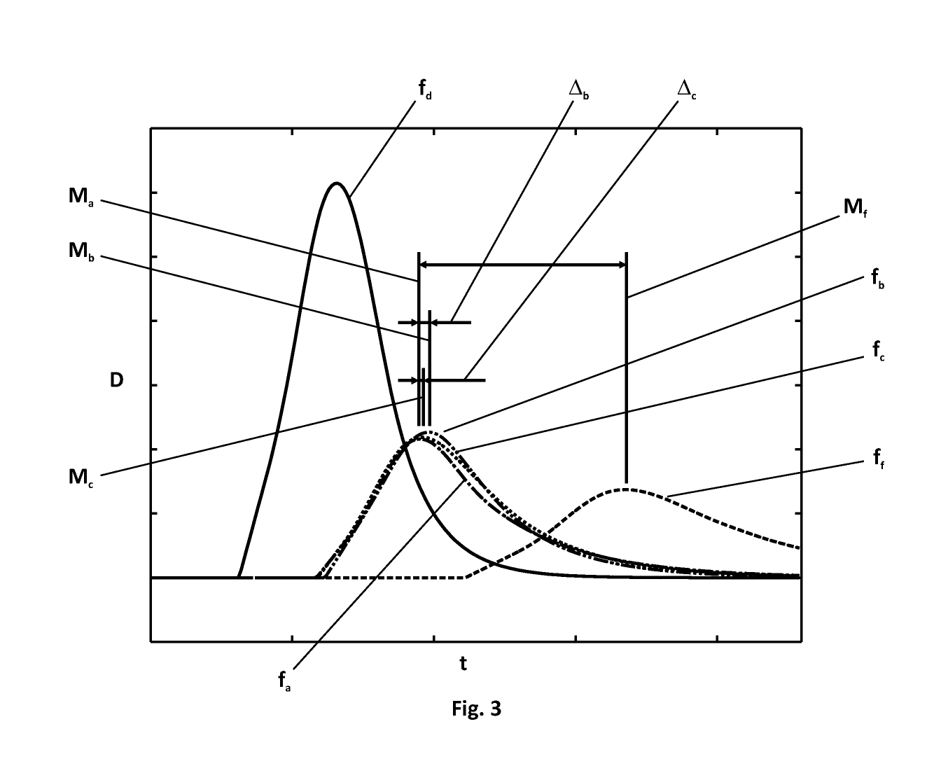

FIG. 3 the density at five locations pursuant to FIG. 1 as an interpolated function of time; and

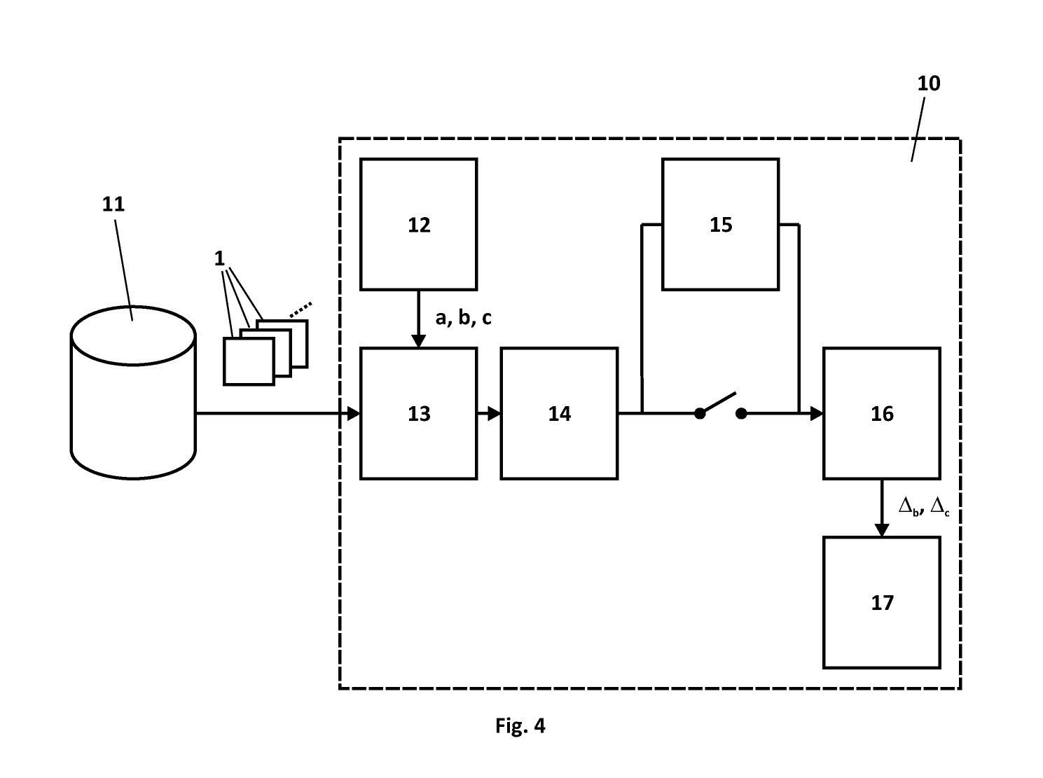

FIG. 4 schematically the construction of a device for performing the method according to the invention.

FIG. 1 illustrates schematically three temporally successive images 1, wherein the images are designated with the respective moment of capture t₁, t₂, t₃. In the foremost image 1 taken at the first time t₁ a sectional view of a human thorax is illustrated schematically directly above the heart. The largest part of image 1 is assumed by the right and left lungs g, h, each of which the corresponding right or left bronchus i, j is assigned to. Between the lungs g, h the ascending aorta e and the descending aorta f as well as the vena cava superior d can be recognized. The recognizable parts of the pulmonary circulation 2, in particular the main trunk 3 of the pulmonary artery (PA) and the right and left pulmonary arteries 4, 5 are of particular interest here, wherein the dotted circles indicate the respectively assigned defined locations (“region of interest”, ROI) in the main trunk a, in the right pulmonary artery b and in the left pulmonary artery c. Furthermore, the section of the breast bone 6 can be recognized at the front side of the thorax. At the opposite rear side the section of the spine 7 is illustrated. The lungs are surrounded by the ribs 8.

In the image 1 illustrated in FIG. 1, the division of the main trunk and/or pulmonary trunk 3 into the two pulmonary arteries 4, 5 can advantageously be recognized, so that the distances between the defined locations in the pulmonary trunk a and in the right pulmonary artery b or in the pulmonary trunk a and in the left pulmonary artery c along the center line of the arteries can be determined from this image 1.

FIG. 2 illustrates schematically a coordinate system with the density D on the axis of ordinates and the time t on the axis of abscissas. In the example illustrated here, a short contrast agent bolus was observed by means of temporally successive images 1 during the passage through the vena cava superior, the pulmonary arteries and the descending aorta. This may, on principle, be performed with any imaging method in which a time-resolved illustration of the vessels is possible and an appropriate contrast agent is available. The progress of the density D is plotted as a function of time t for three locations pursuant to FIG. 1: the initially ascending function F_{d} represents the density D determined in the region of the vena cava superior d, the function Fₐ represents the density D determined at the defined location in the pulmonary trunk a, and the function F_{f} illustrates the progress of the density D in the descending aorta f. The moments of capture t₁, t₂, t₃ of the images indicated in FIG. 1 are drawn in as vertically dashed lines, wherein the illustrated density progresses were obviously determined from more than three images 1.

In the illustrated example the density D corresponds to the contrast agent content in the blood at the respective moments of capture of the images. It is measured and fitted with a suitable method unless the temporal resolution of the individual images is already sufficient to determine the maximum of the contrast agent content with an exactness of approx. 0.1 s. The linearly interpolated functions F_{d}, Fₐ, F_{f} which merely connect the determined values by lines are therefore superimposed with adapted (“fitted”) spline functions representing more realistic, since smoother, interpolations of the density D.

FIG. 3 illustrates a coordinate system according to FIG. 2, wherein here exclusively the curves of the density D interpolated by a spline fit are plotted for different locations pursuant to FIG. 1. In addition to the curves for the vena cava superior f_{d}, the pulmonary trunk fₐ and the descending aorta f_{f} already illustrated in FIG. 2, the curves for the right and left pulmonary arteries f_{b}, f_{c} are also illustrated here. The maximum Mₐ of the density D (corresponds here to the contrast agent maximum) in the pulmonary trunk, the maxima M_{b}, M_{c} of the density D in the right and left pulmonary arteries and the maximum M_{f} of the density D in the descending aorta are plotted as vertical lines. The time difference between the time of the passing of the contrast agent maximum in the main trunk and in the two locations positioned downstream enables, for instance, the diagnosis of pulmonary hypertension (PH). The determined time differences are each plotted as horizontal distances, wherein Δ_{b} designates the time difference between the maximum M_{b} in the right pulmonary artery and the maximum Mₐ in the pulmonary trunk and Δ_{c} designates the time difference between the maximum M_{c} in the left pulmonary artery and the maximum Mₐ in the pulmonary trunk. If at least one of these time differences Δ_{b}, Δ_{c} is larger than approx. 0.5 s, a diagnosis of PH may be made.

If the distance between the ROIs along the center line of the PA can be determined, as described in connection with FIG. 1, it is also possible to determine the velocity of the contrast agent maximum. This also enables the supporting of a diagnosis: If the velocity is below 120 mm/s, this can also indicate PH.

FIG. 4 schematically illustrates the device 10 according to the invention for the processing of images 1 and/or the construction of the device 10. In order to be able to load and process the images 1, the device 10 is usually connected with a data base 11 or a comparable memory. Alternatively, the images 1 may also be transmitted by a direct connection of an imaging device with the processing device 10 illustrated here. On receipt of the images 1, they first of all get to an assigning unit 13 in the device 10. The assigning unit 13 is moreover connected with a selection interface 12 transmitting the provisions for the assignment of at least two defined locations a, b, c to the assigning unit 13. The selection interface 12 may include a configuration memory in which the provisions are stored, or the selection and/or indication of the provisions may be performed interactively via a connection with an image recognition unit or via a user interface. Additionally, the selection interface 12 may include indications about the distances between the selected locations a, b, c which it may also provide to the assigning unit 13.

The assigning unit 13 applies the provisions obtained from the selection interface 12 to all of the received images 1 and is connected with a calculating unit 14, so that the result of the assignment and the distances may be transmitted to the calculating unit 14. The calculating unit 14 calculates the density at the defined locations a, b, c, i.e. it converts the images 1 provided with the defined locations a, b, c to a table of density values which indicates the average density of all the defined locations a, b, c for each image 1, wherein the moments of capture t₁, t₂, t₃ assigned to the images 1 are transferred to the respective density values determined from the images 1. The calculating unit 14 is either connected directly with an analysis unit 16 and/or with an interpolation module 15, so that the determined table is subsequently transmitted jointly with the received distances either directly to the analysis unit 16 or to the interpolation module 15.

The interpolation module 15 adds to the received table additional entries between the entries assigned to the images 1 in that the density values of successive images 1 are interpolated pursuant to a provision stored in the interpolation module 15 or a provision defined otherwise. The table enlarged this way may be transmitted to the analysis unit 16 via a connection between the interpolation module 15 and the analysis unit 16.

The analysis unit 16 processes the table obtained from the calculating unit 14 or the interpolation module 15 such that a time assigned to a maximum Mₐ, M_{b}, M_{c} of the density D at the respective point is stored for each defined location a, b, c. Moreover, the time differences Δ_{b}, Δ_{c} between the maxima of the stored times are determined. By means of the values for the distance between the defined locations a, b, c as transmitted with the table it is moreover possible for the analysis unit 16 to determine flow rate or velocity values from the time differences and to store same.

An output of the analysis unit 16 is connected with an input of a display 17. The display 17 has therefore access to the results of the analysis unit 16. In the display 17 a limit value for the time difference Δ_{b}, Δ_{c} and a limit value for the flow rate may be stored, so that the display 17 cannot just display the time differences Δ_{b}, Δ_{c} obtained from the analysis unit 16, but can also compare same with the limit values and output a signal if the limit value of the time differences Δ_{b}, Δ_{c} is exceeded and the limit value of the flow rate is undercut.

In the framework of a study up to 20 images of the pulmonary artery were made at the level of the trachea splitting with 28 layers each and a resolution of the voxels of approx. 0.6×0.6×0.6 mm. First of all, an image was made without a contrast agent. Then, 20 ml of contrast agent were injected into an arm vein with 5 ml/s, and 4 s after the start of the contrast agent injection the up to 19 images were made with an interval of 1 to 2 s each. The interval depended on the results of a previously performed examination with the right heart catheter and was stopped as soon as the contrast agent had run off the descending aorta so as to avoid unnecessary radiation exposure of the test persons.

The images were reconstructed with a medium hard kernel and were stored anonymized in the form of DICOM files. A layer in which the PA was well recognizable and in which little movement over time could be recognized was selected from the 28 layers. Circular measurement regions (ROIs) were drawn in the main trunk of the PA and in the right and left PAs and the average radiologic attenuation was determined at each time. These values were plotted over time and fitted with a smoothing spline fit (cf. FIG. 2). This was performed with a self-written algorithm in MATLAB.

These curves were used to determine the time differences between the ROIs (cf. FIG. 3). The distance between the ROIs was determined by means of ImageJ in that the ROIs were transferred to a CT of the entire thorax. Subsequently, a layer following the progress of the PA was produced with a multi-planar image and the length of the curve between the ROIs was determined therein. This distance was divided by the respective time difference so as to determine the rate of the contrast agent bolus.

1. A method for processing images of the pulmonary circulation of a living being in order to characterize the arterial blood flow, wherein a plurality of temporally successive images are processed, wherein
a) at least two locations (a, b, c) in an image are defined,
b) the defined locations (a, b, c) are assigned to all images,
c) the density (D) at the defined locations (a, b, c) is determined in all images,
d) the density (D) at the defined locations (a, b, c) is calculated as a function (Fₐ, fₐ, f_{b}, f_{c}) of time (t), and
e) the at least one time difference (Δ_{b}, Δ_{c}) between the maximum values (Mₐ, M_{b}, M_{c}) of the density (D) at the at least two defined locations (a, b, c) as a function of time (t) is analysed and displayed.

2. The method according to claim 1, wherein the images of the pulmonary circulation through substantially cuts of the thorax in the region of the pulmonary trunk (Truncus pulmonalis) and at least one pulmonary artery (Arteria pulmonalis) are used, wherein one defined location (a) in the region of the pulmonary trunk and at least one defined location (b, c) in the region of the at least one pulmonary artery are selected.

3. The method according to claim 1, wherein radiographic images using a contrast agent are used as images, wherein the density (D) of the images at the defined locations (a, b, c) is determined as average radiographic attenuation.

4. The method according to claim 1, wherein magnetic resonance tomography images using a contrast agent are used as images, wherein the density (D) of the images at the defined locations (a, b, c) is determined as average signal and/or as average brightening with suitable weighting.

5. The method according to claim 1, wherein images are processed at an interval between the moments of capture (t₁, t₂, t₃) of at most 5 s.

6. The method according to claim 1, wherein the density (D) for each defined location (a, b, c) is interpolated as a function of time (t) prior to the analysis of the at least one time difference (Δ_{b}, Δ_{c}) between the maximum values (Mₐ, M_{b}, M_{c}) of the density (D), and that wherein the time difference (Δ_{b}, Δ_{c}) between the maximum values (Mₐ, M_{b}, M_{c}) of the interpolated functions (fₐ, f_{b}, f_{c}) is determined.

7. The method according to claim 1, wherein the at least one time difference (Δ_{b}, Δ_{c}) is compared with at least one predetermined limit value and wherein a signal is output if the at least one limit value is exceeded.

8. The method according to claim 1, wherein the distance between the defined locations (a, b, c) is determined and a blood flow rate is determined from the distance and the time difference (Δ_{b}, Δ_{c}).

9. The method according to claim 8, wherein the at least one blood flow rate is compared with at least one predetermined limit value and wherein a signal is output if the at least one limit value is exceeded.

10. A device for processing images of the pulmonary circulation of a living being in order to characterize the arterial blood flow, wherein a plurality of temporally successive images are processed, characterized by
a) a selection interface of at least two defined locations (a, b, c) of an image,
b) a unit for assigning the at least two defined locations (a, b, c) to all images,
c) a unit for determining the density (D) at at least two defined locations (a, b, c) in all images,
d) a unit for calculating the density (D) for the defined locations (a, b, c) as a function (Fₐ, fₐ, f_{b}, f_{c}) of time (t),
e) a unit for analysing the at least one time difference (Δ_{b}, Δ_{c}) between the maximum values (Mₐ, M_{b}, M_{c}) of the density (D) for the at least two defined locations (a, b, c) as a function of time (t), and
f) a display for the at least one time difference (Δ_{b}, Δ_{c}).

11. The image processing device according to claim 10, wherein the images of the pulmonary circulation represent substantially cuts in the region of the pulmonary trunk (Truncus pulmonalis) and at least one pulmonary artery (Arteria pulmonalis), wherein one defined location (a) is arranged in the region of the pulmonary trunk and at least one defined location (b, c) in the region of the at least one pulmonary artery.

12. The image processing device according to claim 10, wherein radiographic images using a contrast agent are provided as images, wherein the unit for determining the density (D) of the images is designed to determine the average radiographic attenuation at the defined locations (a, b, c).

13. The image processing device according to claim 10, wherein magnetic resonance tomography images using a contrast agent are provided as images, wherein the unit for determining the density (D) of the images is designed to determine the average signal and/or the average brightening with suitable weighting at the defined locations (a, b, c).

14. The image processing device according to claim 10, wherein the interval between the moments of capture (t₁, t₂, t₃) of the images is at most 5 s.

15. The image processing device according to claim 10, wherein the unit for analysing the at least one time difference (Δ_{b}, Δ_{c}) between the maximum values (Mₐ, M_{b}, M_{c}) of the density (D) comprises a module for interpolating the density (D) as a function of time (t).

16. The image processing device according to claim 10, wherein the display comprises a signaler for comparing the time difference (Δ_{b}, Δ_{c}) with a predetermined limit value and for outputting a signal if the limit value is exceeded.

17. The image processing device according to claim 10, wherein a unit for determining the distance between the defined locations (a, b, c) and for determining a blood flow rate from the distance and the time difference (Δ_{b}, Δ_{c}) is connected with the unit for analysing the at least one time difference (Δ_{b}, Δ_{c}).

18. The image processing device according to claim 17, wherein the display comprises a signaler for comparing the blood flow rate with a predetermined limit value, and for outputting a signal if the limit value is exceeded.

19. The method according to claim 5, wherein images are processed at an interval between the moments of capture (t₁, t₂, t₃) of between 0.5 and 2 s.

20. The method according to claim 7, wherein the predetermined limit value for the at least one time difference is approx. 0.5 s.

21. The method according to claim 9, wherein the predetermined limit value for the at least one blood flow rate is approx. 120 mm/s.

22. The image processing device according to claim 14, wherein the interval between the moments of capture (t₁, t₂, t₃) of the images is between 0.5 and 2 s.

23. The image processing device according to claim 16, wherein the predetermined limit value for the time difference is approx. 0.5 s.

24. The image processing device according to claim 18, wherein the predetermined limit value for the blood flow rate is approx. 120 mm/s.
